(19) United States
(12) Patent Application Publication
(10) Pub. No.: US 2015/0202016 A1
(43) Pub. Date: Jul. 23, 2015

(54) Impact-Resistant Fastening Assembly for a Medical Device

(71) Applicant: Sanofi-Aventis Deutschland GmbH, Frankfurt am Main (DE)

(72) Inventors: Michael Jugl, Frankfurt am Main (DE); Axel Teucher, Frankfurt am Main (DE)

(73) Assignee: Sanofi-Aventis Deutschland GmbH, Frankfurt am Main (DE)

(21) Appl. No.: 14/424,374

(22) PCT Filed: Aug. 28, 2013
(86) PCT No.: PCT/EP2013/067752

§ 371 Date: Feb. 26, 2015

(30) Foreign Application Priority Data
Aug. 31, 2012 (EP) 12182524.4

(51) Int. Cl. A61B 19/00; A61M 5/24; A61B 5/00; A61M 5/142; A61B 5/145

(52) U.S. Cl. CPC A61B 19/26; A61M 5/14244; A61B 5/14532; A61B 5/6835; A61M 5/24; A61B 5/742; A61B 5/0002; A61B 2019/267; A61B 2560/0443; A61M 2005/14264

(57) ABSTRACT

The present invention relates to a fastening assembly for a medical device and to a respective medical device, wherein the fastening assembly comprises a support structure and a device component releasably fastenable to the support structure by means of mutually corresponding fastening members being adapted to abrogate an interconnection of the device component and the support structure when becoming subject to a force effect or a mechanical impact above a predefined threshold.

### CROSS REFERENCE TO RELATED APPLICATIONS

The present application is a U.S. National Phase Application pursuant to 35 U.S.C. §371 of International Application No. PCT/EP2013/067752 filed Aug. 28, 2013, which claims priority to European Patent Application No. 12182524.4 filed Aug. 31, 2012. The entire disclosure contents of these applications are herewith incorporated by reference into the present application.

### FIELD OF INVENTION

The present invention relates to the field of portable medical devices and in particular to an impact-resistant or shock-proof fastening assembly for a medical device to protect the device and components thereof against mechanical impact.

### BACKGROUND

There exists a variety of portable medical devices such like injection devices or analysis devices, by way of which a patient may conduct self-treatment, in particular self-administration of a medicament.

Especially with patients suffering diabetes, a blood glucose level has to be regularly determined, e.g. by making use of a blood glucose measurement device (BGM). Depending on the measured data and a determined blood glucose concentration the patient may then individually select a dose of a medicament which is to be administered, e.g. by way of injection.

There exists a large variety of medical devices for analysing and treating patients for diagnostic and/or therapeutic purpose. Such medical devices are sometimes rather fragile and sensitive to mechanical impact.

In general, medical devices may comprise a variety of sensitive components which require sufficient protection against external hazards. Portable or mobile medical devices may also comprise a large variety of electronic components, by way of which various functionalities of the device can be configured, controlled and conducted. Additionally, such devices may comprise various input and/or output means, such like a display, operating buttons and knobs, regulators, dose dials and so on.

Moreover, medical devices may also comprise a storage module by way of which repeated use of the medical device can be monitored and logged. Also, medical devices may comprise a communication module, such like an interface, by way of which treatment-related or device-configuration-related data can be exchanged with additional devices, such like personal computers or smartphones.

The various components of such portable medical devices may be rather susceptible to mechanical impact and may affect the general operability of such devices when exposed to mechanical impact above a certain threshold.

It is therefore an object of the present invention to provide an improvement regarding medical device protection in order to enhance robustness and susceptibility of the medical device and its component against mechanical impact. It is a particular aim to protect selected components of a medical device against mechanical impact and to provide a well-defined impact- or crash-behaviour of the medical device. The improvement regarding mechanical impact protection should be rather simple and cost-efficient to realize.

### SUMMARY

In a first aspect a fastening assembly for a medical device is provided. The fastening assembly comprises a support structure and a device component releasably fastenable to the support structure by means of mutually corresponding fastening members. The fastening members of the support structure and the device component are particularly adapted to abrogate an interconnection of the device component and the support structure when becoming subject to a force effect or when becoming subject to a mechanical impact above a predefined threshold.

In general, the fastening assembly with its fastening members provided on the support structure and on the device component is adapted to establish a durable interconnection of the device component and the support structure. In the event that the medical device, for which ever reason, becomes subject to an inadmissibly high mechanical impact or a respective mechanical load, the inter-engaging fastening members of the support structure and the device component release in a well-defined way. The mutually corresponding fastening members thereby support and induce a well-defined abrogation and release of the device component from the support structure.

The inter-engaging fastening members of the support structure and the device component release in a well-defined way when becoming subject to a force effect or a mechanical impact above a predefined threshold. The predefined threshold is selected in a way to ensure the well functioning or general operability of the device by establishing a durable interconnection of the device component and the support structure as long as the mechanical impact or a respective mechanical load or force is below that threshold. The threshold may for example be selected according to regulatory requirements that may define that a device withstands a drop of a certain height. Accordingly, the threshold is selected and the fastening assembly with its fastening members provided on the support structure and on the device component is adapted to establish a durable interconnection of the device component and the support structure. Thereby, the device components stay in place and the device remains fully functioning even after a drop of that certain height.

However, in case the device becomes subject to an inadmissibly high mechanical impact or a respective mechanical load, the inter-engaging fastening members of the support structure and the device component release in a well-defined way. For example, a drop from a bigger height might cause damage to any component of the device and might therefor be considered inadmissible. Although not required from a regulatory perspective, e.g., from a manufacturer's or user's perspective it might beneficial to define the reaction of a device when becoming subject to a force effect or when becoming subject to a mechanical impact above a predefined threshold. Therefore, the disintegration and separation of the respective fastening member into at least two separate pieces as a result to a mechanical impact or a respective mechanical load or force above a threshold provides a well-defined releasing behavior. The inter-engaging fastening members of the support structure and the device component release in a well-defined way. This could be a way to provide improved medical device protection in enhancing robustness and susceptibility of the medical device and its component against mechanical impact.

The threshold may be selected in accordance with considering inadmissibly high impact. The threshold may be selected in accordance with regulatory requirements. The threshold may be selected taking device operability considerations into account. The threshold may be selected according to quality, manufacturing/assembling, and/or usability/handling requirements. The threshold may be selected taking safety buffer considerations into account. The threshold may be selected taking at least of the preceding considerations into account or any combination thereof.

By separating support structure and device component in the event of an undue mechanical impact or mechanical load, the device component can be effectively protected against mechanical impact. By releasing the device component from the support structure during an impact- or crash-scenario, the magnitude and duration of a mechanical impact impinging on the device component can be effectively reduced compared to a configuration, wherein the device component remains unreleasably fastened to the support structure.

In effect, by releasably engaging the support structure and the device component, at least a part of the mechanical energy released in an impact- or crash-scenario can be converted into kinetic energy or into an angular momentum of the device component, which may separate and displace relative to the support structure.

In this way, decelerations typically impinging on a housing of a medical device during an impact- or crash-scenario and respective mechanical peak loads can be transferred to the releasing device component in a damped and less intense way.

In a preferred embodiment, the fastening member of the device component is positively engaged with the fastening of the support structure. By implementing a positive engagement of the mutually corresponding fastening members of the device component and the support structure, an abrogation of the respective interconnection can be non-destructive thereby leaving the mutually corresponding fastening members of the support structure and the device component substantially intact.

In a further embodiment, the fastening member of the device component is adapted to form a clip joint with the fastening member of the support structure. Here, a clip joint is only one of a plurality of examples on how to establish a positive engagement of the fastening members of the device component and the support structure. The clip joint may provide a predetermined removal force above which the interconnection of the device component and the support structure is effectively abrogated.

With a clip-joint-type fastening assembly, a re-connection of the device component and the support structure can be established even when the interconnection has been abrogated due to an undue force effect. In general, and especially with a positive engagement of device component and support structure, the fastening assembly may provide a releasable and re-connectable fastening of the device component and the support structure of the medical device.

In a further embodiment, the fastening members of the device component and the support structure comprise a receptacle or an orifice to engage with a shaft portion to be received therein, wherein the shaft portion further comprises a widened head at a free end. Typically, a diameter of the widened head of the shaft portion is substantially larger than the inner diameter of the receptacle or of a corresponding orifice. Once assembled, the widened head can only be urged out of the receptacle or through the orifice when a tear-off force or a respective impact applies above a given threshold.

In typical embodiments, the support structure comprises the orifice or a correspondingly shaped receptacle having a neck portion to positively engage with a rather straight and elongated shaft having a radially widened head at its free end. Here, the shaft portion with its widened head is provided on or is attached to the device component. Moreover, the shaft portion with its enlarged head section may be integrally formed with the device component.

In other embodiments it is also conceivable, that it is the device component which comprises the receptacle or a corresponding orifice to receive a correspondingly shaped shaft and a respective radially widened head portion at its free end. There, the shaft is preferably arranged on the support structure.

Irrespective on the actual design and embodiment of the mutually corresponding fastening members of support structure and device component it is generally sufficient, when the support structure and the device component each comprise only one fastening member. However, each of the device components and the support structures may also comprise a plurality of fastening members. Here, a separation of the device component from the support structure is only attainable, when all mutually engaging fastening members of support structure and device component release.

By mutually fastening the support structure and the device component by means of a plurality of pairs of inter-engaging fastening members, eventual geometric tolerances of a single fastening member, which may come along with respective variations in terms of a maximum pull-off force, may mutually compensate. In this way, an influence of geometric tolerances of the fastening members of the device component and the support structure can be reduced or may substantially balance out.

Moreover, by varying the mere number of substantially equally configured inter-engaging fastening members, a correspondingly varying magnitude of a maximum bearable mechanical load or impact can be easily designed and realized. Additionally, with a plurality of mutually corresponding fastening members, a durable and reliable mutual fastening of the device component and the support structure can be provided. Even in case that a single fastening member may fail, the device component may still be securely assembled and attached to the support structure.

In a further embodiment, at least one of the fastening members of the device component and/or of the support structure is elastically deformable. By means of a well-defined elastic deformation, a positive engagement of the mutually corresponding fastening members can be abrogated in the event that a force effect above a predefined threshold applies. The elasticity of the at least one fastening member may be particularly designed in order to attain and/or to provide a well-defined releasing behaviour in the event of mechanical impact or other externally applied force effects.

In preferred embodiments at least one of the mutually corresponding and inter-engaging fastening members of the device component and the support structure comprises a plastic material, in particular an injection molded plastic material. For instance, a thermoplastic material exhibiting predefined elastic deformation properties can be used to form at least one of the fastening members of the device component and/or of the support structure. Instead of or additional to thermoplastic materials, the at least one fastening member may at least partially comprise a duro-plastic material or an elastomeric material.

By making use of materials featuring a predefined degree of elasticity and/or deformabilty, the mutually corresponding and inter-engaging fastening members can be manufactured in such a way that their geometric shapes almost exactly match.

Alternatively, it could be also beneficial, when inter-engaging and mutually corresponding fastening members of the support structure and the device component are manufactured within a given tolerance regime in order to facilitate and to provide a well-defined releasing behaviour in response to mechanical loads.

In a further embodiment, at least one of the fastening members of the device component and/or of the support structure comprises a predetermined breaking- or bending structure. This way, instead of releasing the mutual engagement of the fastening members in a non-destructive way, selected fastening members may disintegrate in order to release the device component from the support structure. When implemented as a predetermined breaking structure, e.g. in form of a predetermined breaking point or breaking line, a disintegration and separation of the respective fastening member into at least two separate pieces can be provided.

When implemented as a predetermined bending structure, only parts of the fastening member may disintegrate from the medical device component or from the support structure, thereby allowing a relative displacement or pivoting of the device component and the support structure. Here, a complete detachment of support structure and device component can be effectively prevented.

However, in the event of an undue mechanical impact or force effect, the device component may become subject to some kind of an evasive movement but does not completely separate from the support structure. Hence, even in case of an undue force effect or impact, the partially loosened device component stays within a pre-defined spatial area and remains connected to the support structure.

A predetermined breaking- or bending structure implies that an abrogated interconnection of the device component and the support structure cannot be re-established. An end user of the device equipped with such a fastening assembly is then obliged to seek a customer support, which may check the device functionality and which may resurrect the interconnection in the course of a maintenance procedure.

An intentional and non-reversible abrogation of the interconnection of the device component and the support structure may be beneficial in terms of device- and user safety. Once the interconnection of the device component and the support structure is abrogated, a further use of the device may no longer be possible. Hence, intended abrogation of said interconnection as a consequence of an undue force effect or impact definitely requires to bring the medical device to a customer support in order to check the general operation and functionality of the medical device.

In a further embodiment, the support structure comprises a housing of a medical device or the support structure is part of a housing of the medical device. Moreover, the support structure may comprise or may provide a mounting platform to receive a plurality of device components, wherein the mounting platform is located inside a housing.

In a further embodiment, the device component comprises an electronic component, a display element, a communication module or a cartridge filled with a medicament. By abrogating an interconnection of the device component from e.g. a housing of the medical device, the respective device component is allowed to conduct an evasive movement relative to the housing in order to protect the respective device component from extreme mechanical load, which may typically arise when the medical device drops down to a ground surface and hits the same substantially undamped.

In a further independent aspect also a portable medical device is provided comprising a housing and at least one device component, such like an electronic component, a display element, a communication module or a cartridge filled with a medicament. Moreover, the portable medical device also comprises a fastening assembly as described above. Typically, the housing of the medical device may then serve as a support structure to which the at least one device component is releasably fastened.

Consequently, the support structure of the fastening assembly is integrated in the housing of the medical device. In other words, the housing of the medical device may comprise at least one fastening member to cooperate with a corresponding fastening member of a device component to be fastened thereto.

In still another embodiment, the medical device comprises a drug delivery device, in particular an injection device such like a pen-type injector. In another embodiment, the medical device may comprise an analysis device, such like a blood glucose monitoring device.

Moreover and according to another embodiment, the drug delivery device, in particular the injection device comprises at least one cartridge filled with a medicament to be administered or delivered to a patient, preferably by way of injection.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two 0 sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, two embodiments of the invention will be described by making reference to the drawings, in which:

FIG. 1 schematically illustrates a first embodiment of a fastening assembly in an interconnected configuration and

FIG. 2 shows the fastening assembly according to FIG. 1 when the device component and the support structure are released.

FIG. 3 shows an initial and mutually interconnected configuration of an alternative embodiment featuring a predetermined breaking structure and

FIG. 4 is illustrative of the embodiment according to FIG. 3 after a release of the interconnection of device component and support structure.

### DETAILED DESCRIPTION

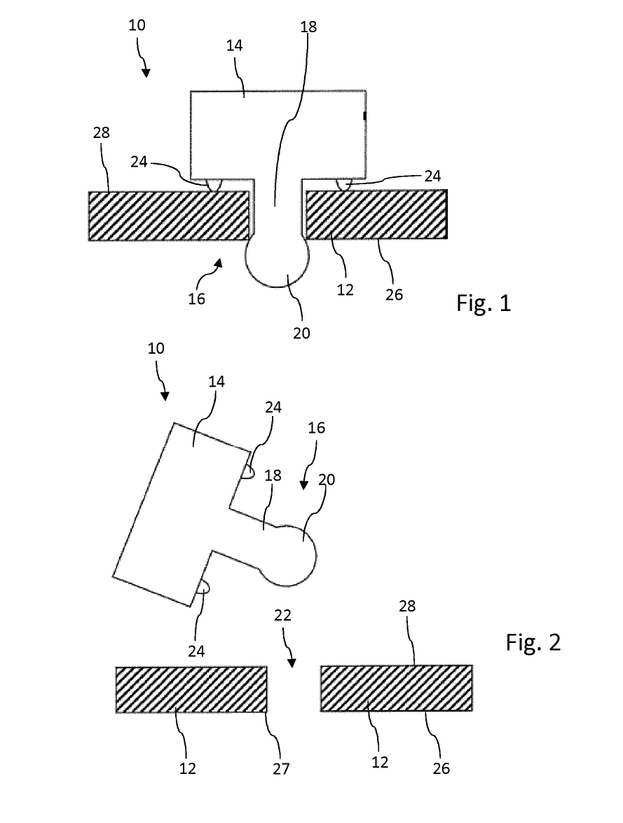

In FIG. 1, an impact-optimized fastening assembly 10 of a medical device is illustrated. The fastening assembly 10 provides a positive engagement of a support structure 12 and a device component 14 of a medical device. The support structure 12 may be integrated into a housing or may even form a housing of a portable medical device, which is not particularly illustrated here. Alternatively, the support structure 12 may provide a mounting platform to receive and to mount a plurality of medical device components 14, wherein a respective assembly of device components 14 and the support structure 12 is located inside a housing of the medical device.

In the embodiment according to FIGS. 1 and 2, the support structure 12 comprises a fastening member 22 featuring an orifice of substantially circular shape. The medical device component 14 comprises a correspondingly shaped fastening member 16 having an elongated shaft portion 18 and a widened head portion 20 at its free end. As illustrated in FIG. 1, a substantially cylindrical portion of the shaft portion 18 extends through the through opening 22 of the support structure 12, wherein the widened head portion 20, which has a diameter exceeding the inner diameter of the through opening 22 extends and protrudes from a lower surface 26 of the support structure 12, which faces away from the device component 14.

In the event that the fastening assembly 10 becomes subject to a force effect or to a mechanical impact above a predefined threshold, the positively engaging interconnection of the two fastening members 16, 22 is released and abrogated, thereby allowing the device component 14 to freely move and to separate from the support structure 12. In this way, the device component 14 may conduct a kind of an evasive movement in order to avoid exposure to extreme mechanical loads, which may otherwise harm or destroy the medical device component 14.

As further illustrated in the embodiments of FIGS. 1 to 4, the medical device component 14 further comprises one or several support elements 24 by way of which the device component 14 abuts with an upper, e.g. inside facing surface 28 of the support structure 12. There may be provided a plurality of spatially distributed and correspondingly separated support elements 24 or there may be only provided a single, e.g. annular shaped support element 24 extending between the upper surface 28 of the support structure and the device component 14. The support element 24 may comprise an elastomeric material, such that the device component 14 may get in tight mechanical contact with the support structure 12.

Moreover, by means of an elastomeric support element 24, the device component 14 may be effectively biased to the support structure 12 thereby keeping the widened head portion 20 in close and tight contact with a lower rim 27 of the through opening 22. The support element 24 is integrally formed with the device component 14 but could also be provided as a separate piece or could be separately attached to the support structure 12.

The embodiment of the fastening assembly 10 as illustrated in FIGS. 1 and 2 provides a releasable and re-engagable fastening of the device component 14 and the support structure 12. Once the device component 14 has been released from the support structure 12, the interconnection as shown in FIG. 1 can be re-established simply by urging the fastening member 16 of the device component 14 through the through opening or orifice 22 of the support structure 12. Here, it is of particular benefit, when at least one of the fastening members 16, 22 comprises at least a pre-defined elasticity.

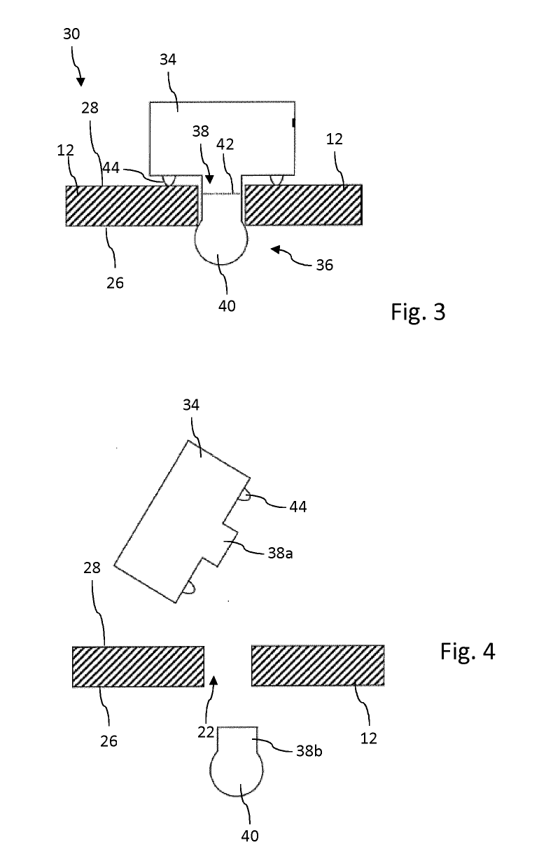

In the embodiment according to FIGS. 3 and 4, the device component 34 comprises a different kind of fastening member 36. From its outer shape, the fastening member 36 is rather identical to the fastening member 16 as illustrated in FIGS. 1 and 2. But the fastening member 36 according to the embodiments as illustrated in FIGS. 3 and 4 comprises a predetermined breaking structure 42, by way of which the shaft portion 38 may disintegrate into an upper shaft portion 38a and a lower shaft portion 38b as shown in FIG. 4 when the fastening assembly 30 becomes subject to an undue force effect or to a respective undue mechanical impact.

Apart from that, the clip joint as illustrated in FIGS. 3 and 4 acts in a similar or identical way as described above in connection with the embodiment as shown in FIGS. 1 and 2. Also here, the shaft portion 38 with its widened head 40 extends through the fastening member 22 of the support structure 12 featuring an orifice that matches and corresponds with the geometric dimensions of the fastening member 36 of the device component 34. Also here, the device component 34 may abut with the support structure by means of at least one support element 44.

However, the fastening assembly 30 as illustrated in FIGS. 3 and 4 does not provide a re-assembly once the interconnection of the device component 34 and the support structure 12 has been abrogated. Instead and in the event of a predetermined breaking of the predetermined breaking structure 42, the respective medical device requires maintenance by a customer support. Hence, with a destructive abrogation of the interconnection of the device component 34 and the support structure 12, a customer or patient making use of the respective medical device is substantially obliged to seek a customer support once the device component 34 has been released from the support structure 12.

1-14. (canceled)

15. A fastening assembly for a medical device, comprising a support structure and at least one device component releasably fastenable to the support structure by means of mutually corresponding fastening members adapted to abrogate an interconnection of the device component and the support structure when becoming subject to a force effect or a mechanical impact above a predefined threshold.

16. The fastening assembly according to claim 15, wherein the fastening member of the device component positively engages with the fastening member of the support structure.

17. The fastening assembly according to claim 15, wherein the fastening member of the device component is adapted to form a clip-joint with the fastening member of the support structure.

18. The fastening assembly according to claim 15, wherein the fastening members of the device component and the support structure comprise a receptacle or an orifice to engage with a shaft portion to be received therein and wherein the shaft portion comprises a widened head portion at a free end.

19. The fastening assembly according to claim 15, wherein at least one of the fastening members of the device component and/or of the support structure is elastically deformable.

20. The fastening assembly according to claim 15, wherein at least one of the fastening members of the device component and/or of the support structure comprises a predetermined breaking- or bending structure.

21. The fastening assembly according to claim 20, wherein the predetermined breaking- or bending structure is configured to provide for disintegration and separation of the respective fastening member into at least two separate pieces thereby releasing the device component from the support structure.

22. The fastening assembly according to claim 16, wherein the support structure comprises a housing of a medical device or wherein the support structure is part of a housing of a medical device.

23. The fastening assembly according to claim 16, wherein the device component comprises an electronic component, a display element, a communication module of a medical device or wherein the device component comprises a cartridge filled with a medicament.

24. The fastening assembly according to claim 16, wherein when becoming subject to a force effect or a mechanical impact above a predefined threshold the fastening members of the support structure and the device component release in a well-defined way.

25. A portable medical device comprising a housing at least one device component and a fastening assembly according to claim 16.

26. The portable medical device according to claim 25, wherein the support structure of the fastening assembly is integrated in the housing of the medical device.

27. The portable medical device according to claim 16 comprising a drug delivery device, an injection device or an analysis device.

28. The medical device according to claim 27, wherein the drug delivery device or the injection device comprises a cartridge filled with a medicament.
